(19) United States
(12) Patent Application Publication
(10) Pub. No.: US 2023/0091910 A1
(43) Pub. Date: Mar. 23, 2023

(54) Engineered System of Stem Cell Rejuvenation to Treat Aging and Disease

(71) Applicant: Centagen, Inc., Boulder, CO (US)

(72) Inventors: Bryant Richard Villeponteau, San Diego, CA (US); Carl A. Fowler, Lakewood, CO (US)

(21) Appl. No.: 17/601,406

(22) PCT Filed: Apr. 2, 2020
(86) PCT No.: PCT/US20/26483

§ 371 Date: Oct. 4, 2021

Related U.S. Application Data
(60) Provisional application No. 62/828,287, filed on Apr. 2, 2019.

(51) Int. Cl. A61K 35/28; C12N 5/0775

(52) U.S. Cl. CPC A61K 35/28; C12N 5/0662; C12N 2500/98; C12N 2500/25; C12N 2501/115; C12N 2501/15

(57) ABSTRACT

The engineered system disclosed herein takes extracted adult stem cells from individual mammal organs (such as adipose fat, bone marrow, or blood) and epigenetically rejuvenates the adult stem cells ex vivo back to a youthful fetal state using chemically defined small molecules. The rejuvenated multipotent stem cells are then exponentially expanded ex vivo in an automated bioreactor under novel conditions that maintain the youthful stem cell function while greatly reducing the risks of producing cancer stem cells. In another variation, this engineered system can also include improving the genetics of autologous adult stem cells using CRISPR base editing of genes affecting all-cause morbidity or mortality or genetic mutations. With or without gene editing, rejuvenated autologous multipotent stem cells are then reintroduced back into an organ or injected systemically into the circulation or bone marrow. The engineered system also includes small molecule treatment of the mammal around the time of stem cell injection to promote tissue regeneration and stem cell engraftment. The engineered system has the potential to promote overall health and longer life expectancy in humans, cats, dogs, and other mammals. The rejuvenated stem cell and small molecule systems offer the potential to treat a wide variety of diseases and disorders such as heart failure, kidney disease, Chronic Pulmonary Disease, frailty, various cancers, rare genetic diseases via CRISPR treated stem cells, and brain diseases such as Alzheimer's, Parkinson's, and vascular dementia. The engineered system treatment with rejuvenated stem cells also has the potential to reverse aging and reduce mortality in humans, dogs, cats, and horses.

### FIELD OF THE DISCLOSURE

The present disclosure relates to reprograming adult stem cells using epigenetic drugs to target a critical number of epigenetic pathways in cultured adult stem cells. The primary goal is to epigenetically reverse age the cells in vitro while significantly minimizing the risks of generating or promoting cancer stem cells. Multipotent stem cells typically undergo programmed epigenetic changes to become progenitor cells that form all the hundreds of differentiated tissues (e.g. neurons, heart muscle, bone marrow) that are required for the development of the functioning organs and tissues in adult mammals. Random epigenetic changes are also induced by the environment: aberrant cell epigenetic changes accumulate with time and stressful environments. Due to both developmental program and environmental influences, epigenetic changes thus proliferate with age to drive a process of cellular senescence that eventually leads to dysfunctional non-dividing senescent cells that can harm surrounding healthy tissue or perversely generate conditions leading to degenerative diseases or tumors that eventually kill the animal. The present system comprises a novel system to take adult stem cells from individual mammal tissues (such as adipose fat, bone marrow, and blood) and reverse age the epigenetic changes in the adult stem cells (rejuvenated stem cells). The disclosure also describes a system to greatly expand these rejuvenated stem cells, while simultaneously mitigating the risks of producing cancer or senescent cells. In another variation, this system can also include improving the genetics of autologous adult stem cells using CRISPR-based editing of genes affecting all-cause morbidity or mortality after reverse aging the cells. With or without gene editing, rejuvenated autologous multipotent stem cells are then reintroduced back into organ systems or injected systemically into the blood or bone marrow. Finally, the system also includes small molecule anti-inflammatory treatment of old mammals to help new stem cell engraftment and tissue regeneration with injection of the rejuvenated stem cells. The overall engineered system goal is to rejuvenate organs and tissues in mammals that are dysfunctional as a result of aging, disease, and injury while avoiding the risks of tumor formation.

### BACKGROUND

Aging involves epigenetic changes to DNA and histones via enzymes that affect the state of methylation or acetylation [1]. These DNA and histone epigenetic changes affect the regulation of genes in the genome in both somatic cells [2, 3] and even adult stem cells [4]. Programed cell changes, lifestyle, stress, and dietary intake can all affect the epigenetic state [5-7], leading to progressive alterations in gene expression as the body ages. Thus, gene expression typically moves away from optimal youthful fitness largely due to the epigenetic drift in genomic structure forged by both programed and environmental influences with the passage of time in adulthood.

The fact that many genes have altered expression with age has led to my hypothesis that a multipath strategy to nudge the expression of many critical genes back toward youthful fitness levels should promote rejuvenation in older animals and return to the low mortality rates of young adults. Since aging plays a major role in age-related diseases and overall health, a composite small-molecule approach targeting critical genes involved in aging could also promote overall health and extend life expectancy. We tested this composite small-molecule supplement hypothesis in Drosophila aging and were successful in significantly extending mean and maximum life span while greatly reducing all-cause mortality [8]. Overall health as measured by enhanced fertility also appeared to be improved by the composite multipath dietary supplement [8].

We further tested this composite small-molecule supplement hypothesis in human clinical trial to see if we could improve markers of overall health and life expectancy. SC100+ is an innovative dietary supplement that targets multiple age-related pathways. The SC100+ was tested in a 15 week human clinical study [9], and this SC100+ clinical revealed striking improvements in critical health markers on SC100+ treatment, including: 1) Significantly reduced blood pressure; 2) Significant increase in “good” HDL Cholesterol; 3) Significantly reduced stress and 4) Significantly improved lung function. Markers of life expectancy (Stress Tolerance, Energy Level, Endurance, Climbing Stairs, and Overall Mood) were also rated as significantly improved after 15 weeks of treatment with SC100+ [10].

The hypothesis that a multipath strategy to nudge the expression of many critical genes back toward youthful fitness levels should promote rejuvenation in older animals and return to the low mortality rates of young adults has also been tested in an animal model of Alzheimer's Disease (AD) to find that AD can be basically prevented and lifespan extended [11]. The same multipath small-molecule strategy was also used in a clinical trial on AD patients and found that cognitive function was stabilized in patients with mild and moderate AD [12, 13].

While these multi-component small-molecule studies appear promising, it is still of limited value: It is difficult to get substantial rejuvenation in vivo in mammals from the small molecule approach, as organ and tissue systems have differing cell types and differentiated states that have been further altered by chronic inflammation, injury, and age-related epigenetic changes. Using the small molecule approach, multiple genes need to be altered without serious side effects. To do this in vivo with all the different cell types (liver, brain, muscle, etc.) that must continue to function normally to sustain the animal's life would be very challenging and highly risky using only small molecule approaches in the whole animal.

One approach to get around the epigenetic changes in older cells is to use heterologous embryonic stem cells from in vitro fertilized human eggs that are pluripotent and can form all tissue types. These embryonic stem cells (ES cells) have very few epigenetic markers and have a functional age of zero. However, there are four significant objections to the use of ES cells in adults: 1) Many people think it is not ethical to use human ES cells; 2) Heterologous ES cells are not genetically the same as the patient and therefore will likely be rejected eventually by the patient's immune system; 3) ES cells can form cancerous teratoma cells; 4) ES cells typically need further programming to generate the tissue-specific progenitor cells that are functionally employed in replacing failing organ-specific cells. Despite more than 20 years of research and development using ES cells, these significant problems have largely restricted ES cells from developing into useful treatments for human regenerative medicine.

A more direct approach to reverse the epigenetic changes in older cells and tissues is to take a sample of adult cells and prepare induced Pluripotent Stem (iPS) cells, which reverts epigenetic changes in adult somatic cells back into their beginning embryonic state. Unfortunately, current iPS methods produce embryonic-like cells that often form tumors [14-16] and need multi-directional programing to form progenitor cells that can replace various damaged tissues [17-19]. As is the case with ES cells, the risks of tumor development and the complicated path to progenitor cells have limited the use of iPS cells in regenerative medicine.

The current disclosure provides a solution to the problems with ES and iPS cells by starting with the patient's own Adult Stem Cells (ASCs) and reverse aging the ASCs to youthful levels, rather than returning the stem cells all the way back to the primitive embryonic iPS state. The system disclosed herein uses controlled reprogramming epigenetic and stem cell expansion systems that prevent the generation and growth of cancer cells or iPS cells. These systems safely reverse age ASCs into multipotent young ASCs. For example, adipose Mesenchymal Stem Cells (MSCs) that are multipotent in forming many differentiated tissue types can be reversed aged back toward the epigenetic state found in the fetal state. Human ASCs can also be expanded a million times or more in an automated bioreactor with specialized media without aging (cell senescence) as is proposed in the current disclosure.

The disclosed system also includes small molecule treatment of the individual mammal to reduce chronic inflammation and to promote tissue regeneration by injected rejuvenated stem cells. The goal is to return an individual's own rejuvenated and expanded adult stem cells back into the organs and tissues of the individual, while minimizing inflammation, senescent cell toxicity, and the risks of generating or promoting cancer cells.

The disclosed system can also be useful in gene editing of human patients with rare genetic diseases. Using viral vectors to edit the all the differing tissues and cells in the human body is challenging. Using the technology system described herein, one can do the gene editing in vitro after reprogramming the ASCs to a rejuvenated fetal state. The rejuvenated gene edited ASCs can next be expanded and then injected back into the patient to treat their specific problem. This system circumvents the need to do gene editing in vivo, which is inherently risky. The system also makes the expansion of edited homologous reversed aged stem cells much more efficient and permits the use of many hundreds of millions of injected edited cells to boost treatment outcomes.

SUMMARY OF THE DISCLOSURE

The current disclosure provides an engineered system for rejuvenating mammalian organs and tissues that are dysfunctional as a result of aging, disease, or injury by epigenetically reprogramming and reverse aging adult stem cells into the fetal stem cell state while minimizing tumor risks, wherein the system has three basic stages: 1) Extract adult stem cells from body and rejuvenate ex vivo using epigenetic reprogramming; 2) Expand Rejuvenated stem cells exponentially; 3) Inject rejuvenated stem cells back into the mammal as a therapeutic treatment.

In the first process stage, one or more Adult Stem Cells (ASCs) are extracted from a body tissue (e.g. adipose, bone marrow, circulated blood, skin, or other organ tissues) using standard systems. The ASCs are partially purified away from blood cells, extraneous tissues, and extracellular debris. The ASCs are then seeded into Vitronectin-treated, feeder-independent cell culture plates with Chemically Defined (CD) and Xeno-free (XF) growth media. The ASCs are treated under novel reprogramming conditions with CD reprogramming drugs that generate an epigenetic reversal to the fetal state (but avoid any reversal to the full iPS embryonic state) via safe epigenetic CD drug reprogramming pathways. No genetic viral DNA or plasmid DNA or RNA vectors are used. The therapeutic active level of reprogramming drugs act on Adenylyl Cyclase (cAMP) Pathway so as to cause a reverse aging of one or more adult stem cells to a fetal stem cell stage. In addition, at least one of the epigenetic pathways from the following group can optionally be added to the Adenylyl Cyclase (cAMP) pathway to promote epigenetic reprogramming efficacy: a) Glycogen Synthase Kinase (GSK) Pathway; b) Rho-associated, coiled-coil containing protein kinase (ROCK) Pathway; c) Protein Kinase C (PKC) Pathway; d) MEK/ERK Pathway. The CD Reprogramming approaches completeness with the generation of Rejuvenated Stem (RS) cells in which most cells simulate a fetal-like Mesenchymal Stem Cell (MSC) gene expression pattern and a methylated DNA age of fetal-like MSC cells as determined by methyl DNA levels (epigenetic age tests).

In the second process stage, RS cells are expanded exponentially in a bioreactor using media containing using one or more of the five epigenetic pathways given above along with anticancer botanical additives to maintain stem cells functionality and to prevent oncogenesis or cellular senescence. The in vitro cell processing will be automated in a closed bioreactor system to minimize costs, infection risks, and human error.

In the third process stage, the expanded RS cells are tested for safety and then injected into the mammal as a therapeutic treatment or frozen in liquid nitrogen for later treatment. Some 50 to 1000 million of the expanded rejuvenated adult stem cells can be injected into a diseased or damaged organ of the mammal to improve organ function or injected systemically into the circulatory system or bone marrow of the mammal to reduce age-related disease and mortality.

In one embodiment of the system disclosed herein, we propose an engineered treatment system for rejuvenating mammalian organs and tissues that are dysfunctional as a result of aging, disease, or injury by epigenetically reprogramming and reverse aging adult stem cells into a fetal stem cell state while minimizing tumor risks, the treatment system comprising: (1) extracting one or more adult stem cells from a mammal and rejuvenating said stem cells epigenetically ex vivo in chemically defined and xeno-free media with therapeutically active levels of drugs which act on the Adenylyl Cyclase (cAMP) Pathway so as to cause a reverse aging of the one or more adult stem cells to a fetal stem cell stage; (2) optionally adding to the Adenylyl Cyclase (cAMP) Pathway at least one of epigenetic pathways: a) Glycogen Synthase Kinase (GSK) Pathway; b) Rho-associated, coiled-coil containing protein kinase (ROCK) Pathway; c) Protein Kinase C (PKC) Pathway; and d) MEK/ERK Pathway to promote epigenetic reprogramming efficacy; (3) exponentially expanding the rejuvenated stem cells in a bioreactor using reprogramming drugs which target the epigenetic pathway(s); (4) and testing the expanded rejuvenated stem cells for safety before injection into a mammal as a therapeutic treatment or freezing in liquid nitrogen for later treatment.

In another alternative embodiment of the system disclosed herein, critical longevity genes in the RS cells can be edited using CRISPR or other gene-editing techniques. One such gene is the oncogene NCORE (a corepressor of histone acetylase), which can be mutated to prevent the inhibition of apoptosis or cell suicide, which helps prevent the generation of metastatic cancer cells and helps kill off dysfunctional senescent cells. Another is the Growth Hormone Receptor (GHR), which is often mutated in men that live over 100 years. Another favored gene mutation is in the Insulin-Like Growth Factor receptor (IGF-R), which is often found in long lived women. The gene-edited RS cells are then expanded under small-molecule conditions that selectively kill cancer and senescent cells. The gene-edited and expanded RS cells are then injected back into the mammal along with small molecule drugs and supplements that promote enhanced stem cell engraftment and tissue regeneration. The gene-edited RS cells could extend human lifespan with these known longevity mutations.

[Para 18] The system described herein can also be useful in gene editing of human patients with rare genetic diseases (e.g. beta-thalassemia or sickle cell anemia). Using viral vectors to edit the human patients' cells in vivo is challenging. Using the technology described herein, one can do the gene editing in vitro using the RS cells that are reversed aged and can be easily expanded to over a billion cells if needed. The gene edited RS cells can then be reinjected back into the patient's blood circulation or bone marrow to gradually replace the patient's mutant cells with gene-edited normal cells.

The current disclosure provides several novel improvements on standard cell reprogramming techniques in that the potential formation of tumors is minimized, while the RS cells, as functional younger cells, should improve their capacity to form progenitor cells that can readily repair or rejuvenate damaged tissues found in the lung, heart, kidney, brain, or muscle. Using the patient's own Adult Stem Cells (ASCs) and reverse aging the ASCs using only chemically-defined, small-molecule epigenetic reprogramming systems in a CD/XF antitumor tissue culture regiment generates the multipotent, safety, and rejuvenation capacity that are inherent in RS cells. The risks of potential tumors and the problematic path to precursor progenitor cells have limited the use of iPS or ES cells in regenerative medicine. The usefulness of the RS cells described herein is also aided by the systems described that enable high volume cell expansion to therapeutically useful numbers (more than 1 billion cells can be generated if needed) in bioreactors without advancing epigenetic age or losing stem cell function due to cellular senescence. As another novel feature of the current disclosure, the potential risks of hidden tumors cells in the rejuvenated and amplified ASCs is dramatically reduced because no iPS cells are formed during reprogramming and the RS cells are expanded in a potent anti-tumor supplemented media. Moreover, most of the in vitro RS cells processing will be automated in a closed bioreactor system to minimize costs, infection risks, and human error.

The RS system also includes oral small molecule treatment before, during, and after ASC injection to reduce both overall chronic inflammation and toxic micro-environments, while enhancing RS cell potential to engraft in various tissues of the older individual. The goal is to reduce chronic inflammation or detrimental age-related conditions that may block or inhibit RS cells from being fully functional on insertion into the circulation or organs of older individuals.

### DETAILED DESCRIPTION OF THE DISCLOSURE

Research over the last 25 years has identified over one hundred genes that are altered with adult aging. These results are fully consistent with the evolution and epigenetic theories of aging, which predict the loss of fitness as a function of age after sexual maturity. The principle underlying idea of this disclosure is that the alterations in the expression of many genes during adult mammalian aging require a potent cell epigenetic treatment strategy to jolt gene expression back toward the youthful adult pattern of expression leading to better health and longer life expectancy. The system details how to epigenetic reprogram and expand human adult stem cells that have a youthful capacity to form multipotent progenitors of diverse types of tissue (mesoderm, endoderm, and epidermal neuronal tissues). Critical to its efficacy, usefulness, and safety, the Chemically Defined (CD) and Xeno Free (XF) epigenetic reprogramming drugs and botanicals described in this disclosure also minimize the potential for generating, promoting, or expanding cancer stem cells.

The present system disclosed herein relates to the reprogramming and safe rejuvenating of adult stem cells (ASCs) that have the capacity to regenerate most tissues and organs in a mammal, while minimizing the overall cancer risks. Mesenchymal Stem Cells (MSCs) are one favored type of adult stem cell typically derived from adipose tissue, bone marrow, circulating blood, and other tissues. MSCs are multipotent mesoderm cells that can regenerate cartilage, skin, muscle, bone, fat cells and even neural cells. Moreover, MSCs are recruited to sites of injury and inflammation, which is a good homing feature of these stem cells in regenerative medicine. Despite the many advantages of MSCs, the present system disclosed herein is not limited to MSC-type stem cells, as a mix of ASCs creates the potential for more balanced regeneration on most tissues and organs. Indeed, pooling ASCs from different sites of extractions (e.g. using both adipose liposuction and circulating blood) could be advantageous.

ASCs can be extracted from a body tissue (e.g. adipose, bone marrow, or circulating blood) using standard procedures [20]. For example, adipose tissues from waist, inner or outer thigh can be extracted using tulip low-pressure, syringe lipo-aspiration systems. The adipose-containing liposuction samples are washed in phosphate-buffered saline (PBS) and centrifuged at 400×Gs for 10 min. to extract the supernatant lipids. The cell-containing lipid fractions are then digested with Collagenase for 25 minutes and centrifuged again to isolate the pelleted stroma vascular fractions (SVF fraction containing ASCs) from the supernatant adipocytes. The SVF fraction is then filtered through a 100-micrometer nylon filter and processed on a density gradient centrifugation using Histopaque-1077 or similar material to isolate the mononuclear cell layer of the SVF fraction [20], which is a crude fractionation of the adipose ASCs.

After the ASCs are partially purified away from extraneous tissues and extracellular debris, ASCs are then seeded into Vitronectin-treated, feeder-independent cell culture plates with Chemically Defined (CD) and Xeno-Free (XF) growth media that sustains adherent ASC growth while suppressing tumor cell formulation. The culture media is critical to the present disclosure, as it must sustain growth of stem cells and be CD and XF, so the expanded ASCs can be approved by the FDA for safe reinjection into the patient without carrying hidden cancer stem cells or triggering foreign tissue immune responses. The tissue culture protocol must also be inexpensive enough to be available to patients that need treatment, which argues against the use of many commercial CD and XF growth media. Surveys of the available data on differing media and actual usage in our lab point to the value of commercial DMEM/F12 supplemented with E8 media [21]. Besides the defined DMEM/F12 components, the standard E8 media includes L-Ascorbic Acid, Selenium, Transferrin, NaHCO3, Insulin, FGF2, and TGF-beta1 or NOTAL [21].

One embodiment of the current disclosure is that the utilized ASC tissue culture media and added drugs/botanicals minimize the risks of cancer cell promotion. Most cancer cells primarily produce energy via high rates of anaerobic glycolysis rather than use aerobic metabolism in mitochondria (the Warburg effect) [22]. As a first step to minimizing cancer cell promotion, anaerobic glycolysis can be reduced by using DMEM/F12 with low glucose (1-5 mM) levels and high levels of TCA cycle precursor sodium pyruvate (1-5 mM) and the fat ketone Beta-hydroxy-methyl butyrate (1-10 mM). To reduce the potential cancer risks using the standard E8 media [21], one embodiment of the disclosure is to add 1-5 mM sodium pyruvate, and 1-10 mM of Beta-hydroxy-methyl butyrate (E8 thus modified is renamed E10).

One disclosed embodiment is a system to reprogram the ASCs back to the fetal state as determined by methyl DNA indexing and by comparison to fetal MSC gene expression pattern. As described above, ASCs are cultured in Vitronectin-treated, feeder-independent cell culture plates with DMEM/F12 plus E10 with 3% oxygen and 5% CO2 at 37 degrees centigrade (CD/XF Culture with E10). When enough ASCs are produced to seed 2 or more dishes or batches of stem cells, the ASCs can then be incubated into DMEM/F12 with E10 medium, wherein the ASCs are treated under controlled small-molecule Epigenetic Reprogramming (EPR) treatment with Forskolin (2-20 μM). In addition, at least one of the EPR drugs CHIR99021 (1-10 μM), Y-27632 (1-10 μM), Gö6983 (1-10 μM), and PD0325901 (0.2-2.0 μM) can optionally be added to promote epigenetic reprogramming efficacy (see Table 1). One novel embodiment of the system disclosed herein is that the ex vivo EPR treatment of individual patient's cells is to generate reversed aged ASCs as monitored by epigenetic methyl-DNA age testing [4, 5, 23] and by a messenger RNA pattern that is similar to fetal MSC gene expression pattern. Moreover, the EPR treatment is engineered so that the cancer-prone iPS cells are never generated. The EPR treated ASCs are tested at 2-3 day intervals post EPR treatment to determine the point that epigenetic age approaches that found in fetal MSC cells and a presumptive rejuvenation stage is obtained. All tissue culture is done in 3% oxygen/5% CO2 culture. At each interval, a small sample of the cells is taken for methyl-DNA (epigenetic age testing) and for messenger RNA profile analysis as a comparison to the messenger RNA profile of fetal stem cells. A successful reprogramming would generate ASCs that have close to zero age (fetal) and a messenger RNA profile like MSCs from fetal adipose, blood, or bone marrow tissues. Successfully reprogrammed ASCs are named Rejuvenated Stem (RS) cells.

Table 1. Reprogramming Targets and Examples of EPR Drugs The Epigenetic Reprogramming (EPR) treatment of the current disclosure is to use therapeutically active doses of the first EPR drug (Forkolin in Table 1) with the optional use of at least one of the other four EPR drugs (CHIR99021, Y-27682, Gö6983, and PD0325901) to promote epigenetic reprogramming efficacy.

| Epigenetic Pathway Targets | Drugs That Act on Targets |
|---|---|
| Adenylyl cyclase (cAMP) | Forskolin |
| Glycogen Synthase Kinase (GSK) | CHIR99021 |
| ROCK 1 and ROCK 2 | Y-27632 |
| Protein Kinase C (PKC) | Gö6983 |
| MEK/ERK | PD0325901 |

Another embodiment of the current disclosure is that the favored reprogramming drugs for some of the Table 1 epigenetic pathway targets can optionally be RNA-based drugs such as RNA interference (RNAi) or antisense oligonucleotide (ASO) therapeutics.

Another embodiment disclosed herein is to exponentially expand the RS cells using hollow fiber bioreactors. Human cells have been grown in hollow fiber bioreactors for many decades and offer advantages as a closed system with better safety from micro-organism contamination and from reduced human error. The hollow fiber media would continue to be CD/XF media with E10 media supplemented with other anti-cancer small molecules described below to expand to about 100 million to 1000 million or more RS cells.

As another embodiment of the disclosure, E10 media is further enhanced for stem cell maintenance and self-renewal during rapid expansion by maintaining some EPR drug treatments using at least one of the EPR drugs at the following doses: 1-3 μM Forskolin and 1-3 μM GSK3 inhibitor CHIR99021, 1-5 μM ROCK inhibitor Y-27632, and 1-5 μM Gö6983 targeting Protein Kinase C (PKC). The PKC drug Gö6983 is also an important biochemical pathways in controlling cancer [24]. The enhanced E10 with the EPR inhibitors is denoted as media E12 in the current disclosure. Thus, stem cell expansion will typically be done in E12 media with at least one EPR drugs.

Another embodiment of the disclosure is to reduce cancer risks during ex vivo ASC expansion by adding a group of 3 to 6 anti-tumor botanicals (Anti-Tumor Additives or ATA) to the E12 media. The components of ATA include at least 3 of the following 6 chemically defined additives: Astragaloside IV, Apigenin, Berberine, Fisetin, Genistein, and Lithium Orotate. All the selected ATA botanicals have shown anticancer activity in multiple published studies. The doses in tissue culture for each of the ATA components comprise: 20 to 100 nM Astragaloside IV; 2 to 10 μM Apigenin; 20 to 100 μM Berberine; 10 to 40 μM Fisetin; 20 to 100 nM Genistein; and 2 to 10 μM Lithium Orotate. These six botanical products kill or slow the growth of cancer cells while having little or no effects on the growth of non-cancerous RS cells. RS cell expansion will typically be done in E12 media with supplemented with the 3-6 antitumor additives (ATA).

Another embodiment of the current disclosure is to use the cell expansion systems above to exponentially expand MSCs from cord blood or any other non-cancerous source other than RS cells. We have successfully expanded adipose Mesenchymal stem cells from a post 60 year old man without reprogramming his stem cells, so the general expansion systems work with aged adult stem cells that have not been rejuvenated. Our cell expansion systems could thus be used to expand Cord Blood MSCs, which do not contain enough stem cells for adults.

As another embodiment of the current disclosure, the final expanded populations of RS cells are tested for methylated DNA age and cancer-free tissue function before injecting back into the mammal. The expanded population of RS cells is tested to ensure that the mean epigenetic age is like fetal MSCs and that the RS cell gene expression profile remains similar to the pattern found in young fetal MSCs from fetal blood, bone marrow, or adipose tissue. The RS cells are also tested for the contamination with cancer stem cells and teratomas by verifying that the RS cells do not form cancer when injected into JAX® Nude Mice. RS cell function is also tested by their ability to form human bone matrix, connective fiber, muscle fiber, kidney tissue, heart muscle, and neurons when injected into the appropriate tissues in JAX® Nude Mice. The RS cells that pass all these quality tests plus tests for bacterial or viral infections can then safely be used clinically in the mammal. Some 0.05 to 1.0 billion cells of the expanded and tested RS cells can be injected into an organ of the mammal to improve organ function or injected systemically into the circulatory system or bone marrow of the mammal to reduce disease conditions or mortality.

As another embodiment of the current disclosure, a sample of the expanded and tested RS cells are frozen in liquid nitrogen in numerous tubes with a million or more RS cells per tube as renewable stocks of RS cells as the mammal ages. Since the RS cells are a valuable autologous stem cell resource that is easily expanded for later use, the RS cells will be banked in frozen storage for use in the decades after their generation. In this respect, frozen RS cell stocks would be like having your own frozen umbilical cord stem cells that have proven to be very helpful to the lucky few whose parents thought to save what is typically thrown away on birthing. However, umbilical cord stem cells often only contain enough stem cells to treat up to a 10-year-old child. Using the general conditions outlined in this disclosure, we have expanded RS cells stocks over a million-fold and 42 passages with no significant increases in epigenetic age as determined by DNA methylation. A frozen vial of the adult stem cells can be thawed and expanded ex vivo to some 1000 million cells under the same ex vivo regiment at 3% oxygen and 5% carbon dioxide in low glucose DMEM/F12 with the same defined and xeno free media containing the anti-tumor additives. RS cell frozen stocks prepared by the specified system in the current disclosure can be a major game changer for employing safe functional fetal-like stem cells to treat many differing conditions even in the very old.

As another embodiment of the current disclosure, longevity genes can be edited into the RS cells using CRISPR or other older gene targeted techniques. One such gene is the oncogene NCORE (a corepressor of histone acetylase), which can be mutated to prevent the inhibition of apoptosis or cell suicide, which helps prevent the generation of metastatic cancer cells and helps kill off dysfunctional senescent cells. Another is the Growth Hormone Receptor (GHR), which is often mutated in men that live over 100 years. Another favored mutated gene is the Insulin-Like Growth Factor receptor (IGF-R), which is often found in long lived women. To take advantage of these beneficial gene modification to a mammal, a mammal's ASCs are rejuvenated into RS cells as described above. Various genes (e.g. NORE, GHR, or IGF-R) in the RS cells are then gene edited using CHRISR or other older gene targeting techniques under the same ex vivo regiment of low glucose DMEM/F12 with E10 media. The edited RS cells are then cultured for 7 to 21 days in DMEM/F12 with E12 media and individual RS cell clones isolated and assayed to check for correct gene editing. The RAN cell clones with the correct gene edits are expanded exponentially in the hollow fiber bioreactors as described above.

In another embodiment of the disclosure, the systems herein can also be useful in gene editing of human patients with rare genetic diseases. Using viral vectors to edit the human patients' cells in the body is challenging. Using the RS system technology described herein, one can do the gene editing in vitro using CRISPR after reprogramming the ASCs to RS cells. The edited RS cells that removes the genetic mutation are then cultured in DMEM/F12 and individual RS cell clones isolated and assayed to check for correct mutational editing. The RS cell clones with the correct gene edits are then expanded in the hollow fiber bioreactors as described above. The mutant-edited RS cells can then be injected back into the patient to treat their specific genetic disease. The genetic edited RS system circumvents the need to do gene editing of the patient's cells in vivo, which is inherently risky. Using the techniques in the current disclosure also benefits systems that use gene editing in vitro, as the patient's edited RS cells can be cloned easily and expanded greatly before injection into the patient. In this case, multiple injection treatments with rejuvenated fully functional RS cells should greatly increase the chances of success with in vitro edited RS cells.

In another embodiment of the disclosure, RS cells are prepared from noncancer stem cells of a cancer patient and the RS cells can be re-injected into the circulation or bone marrow after chemotherapy or radiation therapy. The option to prepared RS cells before chemotherapy or radiation therapy begins and then inject the RS cells back into the patient's after each round of chemotherapy or radiation therapy is attractive for those with cancer, as stem cell transplants after chemotherapy and/or total body irradiation often improve the survival rate of cancer patients. After high dose chemotherapy, the patient receives the stem cell transplants to quickly rebuild the immune system. While bone marrow stem cell transplants have been used for decades in the treatment of cancer, injection of RS cells into a patient's bone marrow should be far superior to the normal adult stem cell treatments that are now used, as RS cells will provide higher doses and better potency.

Chemotherapy or total body radiation can also create space for new bone morrow engraftment of the newly injected RS cells. If the RS cells are to be injection locally into a particular organ or tissue area, then the organ or tissue area could be specifically targeted with radiation prior to the injection of the RS cells into that specific tissue. Note that there are long-term side effects of chemo and total body irradiation such as higher risks of later cancer, heart problems, reduced lung capacity, and kidney, bone and joint problems. However, most of these problems could be reversed or improved by injection of the RS cells. For example, total body irradiation of old mice followed by transplantation of young bone marrow stem cells preserves memory in old mice and may have long term positive effects on aging [25]. Nevertheless, the use the total body irradiation or potent chemotherapy drugs on healthy individuals not suffering from cancer or a deadly disease are currently too risky without careful research showing specific protocols of chemotherapy or total body irradiation that provide significant long-term efficacy and safety in animals and human clinical trials.

In another embodiment of the disclosed system, the system disclosed herein also includes small molecule treatment of the mammal before, during, and after RS cells injection to reduce both overall chronic inflammation and toxic micro-environments, while enhancing stem cell potential to engraft in various organs of the older individual. The goal is to reduce chronic inflammation or detrimental age-related conditions that may block or inhibit rejuvenated RS cells from being fully functional on injection into the older individual due to circulating toxic factors from senescent cells or failing kidneys or liver. One favored embodiment of the disclosure is a system for promoting stem cell regeneration of mammalian organs and tissues, wherein therapeutic effective doses of an inhibitor of Protein Tyrosine Phosphatase 1B are co-injected into mammalian organs, veins, or bone marrow along with effective doses of RS cells. The Protein Tyrosine Phosphatase 1B inhibitor drug MSI 1436 is known to favor regeneration of organs and tissue [26, 27] and should recruit and help engraft the RS cells to the most useful tissues and organs. The therapeutic dose of MSI 1436 is about 0.3 mg/kg to 30 mg/kg (weight/weight).

In another embodiment of the disclosure, the patient is treated before and after RS cell injection with stem-cell-promoting dietary supplements. For example, a favored system for promoting stem cell engraftment of transplanted RS cells and promotion of tissue regeneration is to give mammals two months of daily oral anti-inflammatory treatment with therapeutically effective amounts of one of the following: (a) Commercially available 10 component SC100+ [9], which contains: Astragalus membranaceus bark extract, Vaccinium Uliginosum fruit extract, Rhodiola Rosea root, Tulsi leaf, Pine Bark OPCs, L-Theanine, Genistein, Vitamin D3, Methyl-Folate, and Vitamin Methyl-B12; or (b) Commercially available dietary supplements containing at least 6 of the following 14 nutraceuticals: Astragalus membranaceus bark extract, pterostilbene, Rhodiola Rosea root, Pine Bark OPCs, L-Theanine, Genistein, Vitamin D3, Methyl-Folate, fisetin, Berberine, Ginger root, CoQ10, Silybum Marranum seed, and Scutelloria baicalensis root. With respect to the current system disclosed herein, these novel dietary formulations are used as oral anti-inflammatory treatments for 4 weeks before and 4 weeks after stem cell injection in the expectation that the dietary supplement cotreatments will promote RS cell engraftment and in vivo tissue regeneration.

Even aging itself may be partly or wholly overcome using the youthful RS cells described herein. In one embodiment of the disclosed system, the RS cells are injected into a mammal's bone marrow and general circulation in several injections over a many week period along with simultaneous oral anti-inflammatory supplements to promote anti-aging rejuvenation and regeneration in mammalian adults of any age. The RS cell system is proposed as an antiaging treatment for mammals such as humans, dogs, cats, and horses.

The RS cell systems described herein should provide the ability to grow an unlimited number of fully functional autologous RS cells from any mammal, which could then be used for treating and curing many chronic and genetic diseases. These RS cells should be able to regenerate most, if not all, organs and tissues in the body. While the potential that stem cells could revolutionizing medicine have long been claimed, clinical trials with stem cells have typically been disappointing. Many technical problems needed to be solved before the full potential of stem cells could be realized. The current disclosure provides the needed technology that will eventually permit rejuvenated stem cells to fulfil their full potential. In other embodiments of the disclosure, the said RS cells provide stem system to treat a wide variety of diseases and disorders such as Cancer, Immune Disorders, Cardiovascular Disease (e.g. Heart Failure), Chronic Obstructive Pulmonary Disease, Frailty, rare genetic diseases via CRISPR treated stem cells, and dementia diseases such as Alzheimer's, Parkinson's, and Vascular Dementia.

In one disease indication, injection of autologous RS cells into a patient's bone marrow can provide immune-deficiency patients with a huge boost to their immune system whether their immunodeficiency is caused by primary immunodeficiencies or old age. For example, potent RS cells will kill the patient's cancer cells far better than the small numbers of functionally weak stern cells in older patients. As the number of immune cells and their function declines with age, RS cells should reinvigorate the patient's immune system. Moreover, if the patient has inherited a genetic mutation causing their immunodeficiency, their immunodeficiency can be corrected with gene editing of the RS cells. In another embodiment of the disclosure, injection of autologous RS cells (some 50 million to 1000 million cells) into the bone marrow is proposed as a treatment for Immunodeficiency diseases.

As another disease indication, around 5.5 million American adults have heart failure and their treatment cost an estimated $30.7 billion in 2012—See Heart Failure—CDC and American Heart Association. Heart Failure has often led to heart transplantation, which is a difficult and expensive treatment. Stem cells offer a potential cure [28]. In another embodiment of the disclosure, RS cells could be injected into and around the damaged heart as a treatment for heart failure.

As another disease indication, some 50 million people live with dementia with a global cost of over $800 billion. Despite more than a thousand clinical trials for Alzheimer's Disease (AD), there is no cure. Stem cell therapy for AD has enormous promise [29, 30]. RS cells could provide the needed stern cells to reverse Alzheimer's and other dementia diseases. Given that dementia typically involves neuronal cell death, stern cell injection to regenerate fully functioning neurons may be the only possible cure. In another embodiment of the disclosure, injection of autologous RS cells into the brain is proposed as potential treatments for dementia diseases such as Alzheimer's, Parkinson's, and vascular dementia.

As another disease indication, kidney disease is a chronic disease leading to great expense and time spent in kidney dialysis (up to $80,000 per year) or finally organ transplantation if a tissue-matched kidney transplant becomes available. Stem cells are potentially a cure by injecting stem cells [31, 32] or creating an artificial kidney using stem cells. In another embodiment of the disclosure, injection of RS cells into and around the kidney is proposed as a potential treatment for kidney disease.

As another disease indication, Chronic Obstructive Pulmonary Disease (COPD) typically arises from damage to the lung due to emphysema or chronic bronchitis. Common causes are cigarette smoking, pollution, infectious diseases, and genetics. While treatment with drugs can help with symptoms, COPD is incurable with current medical treatments. This is a common chronic disease with some 3 million cases in the US per year. Stem cells in general and MSCs in particular may provide real cures [33, 34]. In another embodiment of the disclosure, injection of autologous RS cells is proposed as a treatment for COPD with much higher efficacy than allogenic MSC cells.

Frailty is an age-related syndrome indicated by a progressive decline in health, endurance, and functional capacity along with muscle weakness and/or sarcopenia. Frailty puts individuals at high risks of falls, chronic disability, hospitalization, and death. Current intervention typically includes exercise and dietary changes with only modest benefits. Since depletion of stem cells and regenerative capacity are a major hallmark of frailty, allogenic MSCs have been tested with some positive clinical benefits [35, 36]. In another embodiment of the disclosure, injection of autologous RS cells into the general circulation or bone marrow is proposed as a treatment for frailty and muscle weakness, which should have higher efficacy than allogenic MSC cells.

There are far fewer transplant organs (liver, heart, lungs, and kidneys) than needed and the transplanted organ are not autologous and thus the patient has to take immune rejection drugs for life. In another embodiment of the disclosure, injection of RS cells into ex vivo damaged or decellularized organs could regenerate autologous organs for implantation into humans in need of an organ transplant.

### REFERENCES

1. Villeponteau B: The heterochromatin loss model of aging. Exp Gerontol 1997, 32:383-394.
2. Walker M P, LaFerla F M, Oddo S S, Brewer G J: Reversible epigenetic histone modifications and Bdnf expression in neurons with aging and from a mouse model of Alzheimer's disease. Age (Dordr) 2013, 35:519-531.
3. Ravaioli F, Bacalini M G, Franceschi C, Garagnani P: Age-Related Epigenetic Derangement upon Reprogramming and Differentiation of Cells from the Elderly. Genes (Basel) 2018, 9.
4. Przybilla J, Rohlf T, Loeffler M, Galle J: Understanding epigenetic changes in aging stem cells—a computational model approach. Aging Cell 2014, 13:320-328.
5. Spolnicka M, Pospiech E, Adamczyk J G, Freire-Aradas A, Peplonska B, Zbiec-Piekarska R, Makowska Z, Pieta A, Lareu M V, Phillips C, et al: Modified aging of elite athletes revealed by analysis of epigenetic age markers. Aging (Albany N.Y.) 2018, 10:241-252.
6. Lawn R B, Anderson E L, Suderman M, Simpkin A J, Gaunt T R, Teschendorff A E, Widschwendter M, Hardy R, Kuh D, Relton C L, Howe L D: Psychosocial adversity and socioeconomic position during childhood and epigenetic age: analysis of two prospective cohort studies. Hum Mol Genet 2018.
7. Najar R A, Wani N A, Bhat J A, Dar N J, Rahat B, Gupta A P, Kaur J, Kaur J, Hamid A: Modulation of dietary folate with age confers selective hepatocellular epigenetic imprints through DNA methylation. J Nutr Biochem 2018, 53:121-132.
8. Villeponteau B, Matsagas K, Nobles A C, Rizza C, Horwitz M, Benford G, Mockett R J: Herbal supplement extends life span under some environmental conditions and boosts stress resistance. PLoS One 2015, 10:e0119068.
9. Villeponteau B, Still Y L: Treating multiple antiaging pathways improves health markers in open label clinical study. Translational Medicine Communications 2017, 2.
10. Villeponteau B: Multipath herbal nutraceutical improves self-reported markers of health and life expectancy in clinical trial. SF J Herb Med 2018, 2.
11. Matsagas K, Rizza C, Goertzel B, Benford G, Villeponteau B: Multipath natural product supplement suppresses dementia symptoms in Amyloid-beta and tau transgenic Drosophila. J Biol Med Res 2018, 2.
12. Villeponteau B, Rizza C: Multipath nutritional supplement for memory, cognition, and coordination US Patent 9,744,2042017.
13. Matsagas K, Villeponteau B, Shankle W, Cruise R, Morales S, Goertzel B, Simmon V, Benford G, Rizza C: Botanical mixture stabilizes cognitive function in patients with mild and moderate Alzheimer's Disease. J Clin Med and Ther 2018, 3.
14. Lin S L, Ying S Y: Mechanism and Method for Generating Tumor-Free iPS Cells Using Intronic MicroRNA miR-302 Induction. Methods Mol Biol 2018, 1733:265-282.
15. Menendez S, Cam us S, Herreria A, Paramonov I, Morera LB, Collado M, Pekarik V, Maceda I, Edel M, Consiglio A, et al: Increased dosage of tumor suppressors limits the tumorigenicity of iPS cells without affecting their pluripotency. Aging Cell 2012, 11:41-50.
16. Trosko J E: Induction of iPS cells and of cancer stem cells: the stem cell or reprogramming hypothesis of cancer? Anat Rec (Hoboken) 2014, 297:161-173.
17. Xu M, Shaw G, Murphy M, Barry F: Induced Pluripotent Stem Cell-Derived Mesenchymal Stromal Cells Are Functionally and Genetically Different From Bone Marrow Derived-Mesenchymal Stromal Cells. Stem Cells 2019.
18. Cypris O, Frobel J, Rai S, Franzen J, Sontag S, Goetzke R, Szymanski de Toledo M A, Zenke M, Wagner W: Tracking of epigenetic changes during hematopoietic differentiation of induced pluripotent stem cells. Clin Epigenetics 2019, 11:19.
19. Nishihara K, Shiga T, Nakamura E, Akiyama T, Sasaki T, Suzuki S, Ko M S H, Tada N, Okano H, Akamatsu W: Induced Pluripotent Stem Cells Reprogrammed with Three Inhibitors Show Accelerated Differentiation Potentials with High Levels of 2-Cell Stage Marker Expression. Stem Cell Reports 2019, 12:305-318.
20. TSEKOURAS A, MANTAS D, TSILIMIGRAS D, MORIS D, KONTOS M, ZOGRAFOS C G: Comparison of the Viability and Yield of Adipose-Derived Stem Cells (ASCs) from Different Donor Areas. In Vivo 2017, 31:1229-1234.
21. Chen G, Gulbranson D, Hou Z, Bolin J, Ruotti V, Probasco M, Smuga-Otto K, Howden S, Diol N, Propson N, et al: Chemically defined conditions for human iPSC derivation and culture. Nature Methods 2011, 8:424-429.
22. Liberti M V, Locasale J W: The Warburg Effect: How Does it Benefit Cancer Cells? Trends Biochem Sci 2016, 41:211-218.
23. Marioni R E, Harris S E, Shah S, McRae A F, von Zglinicki T, Martin-Ruiz C, Wray N R, Visscher P M, Deary I J: The epigenetic clock and telomere length are independently associated with chronological age and mortality. Int J Epidemiol 2018, 47:356.
24. Cooke M, Magimaidas A, Casado-Medrano V, Kazanietz MG: PROTEIN KINASE C IN CANCER: THE TOP FIVE UNANSWERED QUESTIONS. Mol Carcinog 2017, 56:1531-1542
25. Das M M, Godoy M, Chen S, Moser V A, Avalos P, Roxas K M, Dang I, Yanez A, Zhang W, Bresee C, et al: Young bone marrow transplantation preserves learning and memory in old mice. Commun Biol 2019, 2:73.
26. Smith A M, Maguire-Nguyen K K, Rando T A, Zasloff M A, Strange K B, Yin V P: The protein tyrosine phosphatase 1B inhibitor MSI-1436 stimulates regeneration of heart and multiple other tissues. NPJ Regen Med 2017, 2:4.
27. Revuelta-Cervantes J, Mayoral R, Miranda S, Gonzalez-Rodriguez A, Fernandez M, Martin-Sanz P, Valverde A M: Protein Tyrosine Phosphatase 1B (PTP1B) deficiency accelerates hepatic regeneration in mice. Am J Pathol 2011, 178:1591-1604.
28. Yamada S, Arrell D K, Rosenow C S, Bartunek J, Behfar A, Terzic A: Ventricular remodeling in ischemic heart failure stratifies responders to stem cell therapy. Stem Cells Transl Med 2020, 9:74-79.
29. Boese A C, Hamblin M H, Lee J P: Neural stem cell therapy for neurovascular injury in Alzheimer's disease. Exp Neurol 2020, 324:113112.
30. Zhang F Q, Jiang J L, Zhang J T, Niu H, Fu X Q, Zeng L L: Current status and future prospects of stem cell therapy in Alzheimer's disease. Neural Regen Res 2020, 15:242-250.
31. Yun C W, Lee S H: Potential and Therapeutic Efficacy of Cell-based Therapy Using Mesenchymal Stem Cells for Acute/chronic Kidney Disease. Int J Mol Sci 2019, 20.
32. Zhao L, Hu C, Zhang P, Jiang H, Chen J: Melatonin preconditioning is an effective strategy for mesenchymal stem cell-based therapy for kidney disease. J Cell Mol Med 2020, 24:25-33.
33. Kokturk N, Yildirim F, Gulhan P Y, Oh Y M: Stem cell therapy in chronic obstructive pulmonary disease. How far is it to the clinic? Am J Stem Cells 2018, 7:56-71.
34. Akram K M, Patel N, Spiteri M A, Forsyth N R: Lung Regeneration: Endogenous and Exogenous Stem Cell Mediated Therapeutic Approaches. Int J Mol Sci 2016, 17.
35. Sun X L, Hao Q K, Tang R J, Xiao C, Ge M L, Dong B R: Frailty and Rejuvenation with Stem Cells: Therapeutic Opportunities and Clinical Challenges. Rejuvenation Res 2019, 22:484-497.
36. Tompkins B A, DiFede D L, Khan A, Landin A M, Schulman I H, Pujol M V, Heldman A W, Miki R, Goldschmidt-Clermont P J, Goldstein B J, et al: Allogeneic Mesenchymal Stem Cells Ameliorate Aging Frailty: A Phase II Randomized, Double-Blind, Placebo-Controlled Clinical Trial. J Gerontol A Biol Sci Med Sci 2017, 72:1513-1522.

1. An engineered system for rejuvenating mammalian organs and tissues that are dysfunctional as a result of aging, disease, or injury by epigenetically reprogramming and reverse aging adult stem cells into a fetal stem cell state while minimizing tumor risks, the system comprising:
one or more adult stem cells extracted from a mammal and rejuvenated epigenetically ex vivo in chemically defined and xeno-free media with therapeutically active levels of drugs which act on the epigenetic Adenylyl Cyclase (cAMP) Pathway so as to cause a reverse aging of the one or more adult stem cells to a fetal stem cell stage;
wherein at least one of epigenetic pathways: a) Glycogen Synthase Kinase (GSK) Pathway; b) Rho-associated, coiled-coil containing protein kinase (ROCK) Pathway; c) Protein Kinase C (PKC) Pathway; and d) MEK/ERK Pathway can optionally be added to the Adenylyl Cyclase (cAMP) Pathway to promote epigenetic reprogramming efficacy;
wherein the rejuvenated stem cells are then expanded exponentially in a bioreactor using one or more reprogramming drugs which target the epigenetic pathway(s); and
wherein the expanded rejuvenated stem cells are tested for safety and then injected into the mammal as a therapeutic treatment or frozen in liquid nitrogen for later treatment.

2. The system of claim 1, wherein the adult stem cells are extracted from a body tissue such as adipose tissue, bone morrow, circulating blood, skin, or other organ tissues, and cultured ex vivo in chemically defined and xeno-free media at about 3% oxygen and about 5% carbon dioxide.

3. The system of claim 1, wherein the chemically defined and xeno-free media comprises low glucose DMEM/F12 with pyruvate, beta-hydroxy-methyl butyrate, L-Ascorbic Acid, Selenium, Transferrin, NaHCO3, Insulin, FGF2, and TGF-betal or NOTAL (E8 Medium), with the further addition of about 1-5 mM sodium pyruvate, and about 1-10 mM of Beta-hydroxy-methyl butyrate (E10 Medium).

4. The system of claim 1, wherein after epigenetic reprogramming, the rejuvenated stem cells can optionally be genetically edited using standard gene editing techniques such as CRISPR to edit known longevity genes such as the oncogene NCORE (a corepressor of histone acetylase), the growth hormone receptor, and the insulin-like growth factor receptor, or mutant genes in human patients with rare genetic diseases.

5. The system of claim 1, wherein the epigenetic pathways further comprise reprogramming drugs and dosages as follows: a) Adenylyl cyclase (cAMP) targeted by about 2-20 μM Forskolin; b) Glycogen Synthase Kinase (GSK) targeted by about 1-10 μM CHIR99021; c) Rho-associated, coiled-coil containing protein kinase (ROCK) targeted by about 1-10 μM Y-27632; d) Protein Kinase C (PKC) targeted by about 1-10 μM Gö6983; and e) MEK/ERK targeted by about 0.2-2.0 μM PD0325901.

6. The system of claim 1 wherein said reprogramming drugs for at least one of the epigenetic pathways optionally comprise RNA-based drugs such as RNA interference (RNAi) or antisense oligonucleotide (ASO) therapeutics.

7. The system of claim 1, wherein the epigenetic rejuvenation into fetal stem cells approaches completion when the stem cells reach a methylated DNA age of fetal-like mesenchymal stem cells as determined by methyl DNA levels (epigenetic age tests) and by messenger RNA patterns that are similar to fetal-like, mesenchymal stem-cell-related gene expression pattern.

8. The system of claim 1, wherein said bioreactor further comprises an automated hollow fiber device operated under conditions that preserve stem cell multipotency and self-renewal while minimizing cell differentiation, cell senescence, and tumor cell promotion, thereby causing the rejuvenated stem cells to be expanded ex vivo to about 50 million to about 1000 million or more rejuvenated stem cells in chemically defined and xeno-free E12 media at about 3% oxygen and about 5% carbon dioxide containing at least 3 out of the following 6 antitumor additives (ATAs): 20 to 100 nM Astragaloside IV; 2 to 10 μM Apigenin; 20 to 100 μM Berberine; 10 to 40 μM Fisetin; 20 to 100 nM Genistein; and 2 to 10 μM lithium Orotate.

9. The system of claim 8, wherein one or more reprograming drugs can be added at somewhat lower doses during the expansion stage of the rejuvenated stem cells to maintain multipotency and self-renewal of the rejuvenated stem cells: 1-3 μM cAMP inhibitor Forskolin, 1-3 μM GSK3 inhibitor CHIR99021, 1-5 μM ROCK inhibitor Y-27632, and 1-5 μM Gö6983 targeting the Protein Kinase C (PKC) along with normal dosage of at least 3 ATAs.

10. The system of claim 1, wherein prior to injection into a mammal or storing in liquid nitrogen the expanded rejuvenated adult stem cells can be retested for methylated DNA age, cancer potential, and stem cell function.

11. An engineered treatment system for rejuvenating mammalian organs and tissues that are dysfunctional as a result of aging, disease, or injury by epigenetically reprogramming and reverse aging adult stem cells into a fetal stem cell state while minimizing tumor risks, the treatment system comprising:
extracting one or more adult stem cells from a mammal and rejuvenating said stem cells epigenetically ex vivo in chemically defined and xeno-free media with therapeutically active levels of drugs which act on the Adenylyl Cyclase (cAMP) Pathway so as to cause a reverse aging of the one or more adult stem cells to a fetal stem cell stage;
optionally adding to the Adenylyl Cyclase (cAMP) Pathway at least one of epigenetic pathways: a) Glycogen Synthase Kinase (GSK) Pathway; b) Rho-associated, coiled-coil containing protein kinase (ROCK) Pathway; c) Protein Kinase C (PKC) Pathway; and d) MEK/ERK Pathway to promote epigenetic reprogramming efficacy;
exponentially expanding the rejuvenated stem cells in a bioreactor using reprogramming drugs which target the epigenetic pathway(s); and
testing the expanded rejuvenated stem cells for safety before injection into a mammal as a therapeutic treatment or freezing in liquid nitrogen for later treatment.

12. The treatment system of claim 11, further comprising injecting the expanded rejuvenated adult stem cells into a diseased or damaged organ of a mammal to improve organ function.

13. The treatment system of claim 11, further comprising injecting the expanded rejuvenated adult stem cells systemically into the circulatory system or bone marrow of a mammal to reduce tissue and organ dysfunction due to aging, age-related disease, or injury.

14. The treatment system of claim 11, further comprising, prior to injection of rejuvenated adult stem cells, giving a mammal at least 4 weeks of daily oral treatment with an anti-inflammatory botanical supplement that promotes in vivo stem cell engraftment and function.

15. The treatment system of claim 11, further comprising injecting the expanded rejuvenated adult stem cells into the bone marrow of a mammal and general circulation in several injections for a multiple week period along with simultaneous oral anti-inflammatory supplements to promote anti-aging rejuvenation and regeneration in mammalian adults.

16. The treatment system of claim 11, further comprising injecting the expanded rejuvenated adult stem cells into the bone marrow of a mammal and general circulation in several injections for a multiple week period along with simultaneous oral anti-inflammatory supplements as a treatment for cancer.

17. The treatment system according to claim 11, further comprising injecting the expanded rejuvenated adult stem cells into the bone marrow of a mammal and general circulation along with simultaneous injections of 0.3 mg/kg to 30 mg/kg (weight/weight) doses of the Protein Tyrosine Phosphatase 1B inhibitor drug MSI 1436 to help promote regeneration and rejuvenation of the mammal.

18. The treatment system of claim 11, further comprising injecting the rejuvenated adult stem cells into and around a damaged heart as a treatment for heart failure.

10. The treatment system of claim 11, further comprising injecting the rejuvenated adult stem cells into and around a brain as a treatment for dementia diseases such as Alzheimer's, Parkinson's, and vascular dementia.

20. The treatment system of claim 11, further comprising injecting the rejuvenated adult stem cells into and around a damaged kidney as a treatment for kidney dysfunction.

21. The treatment system of claim 11, further comprising injecting the rejuvenated adult stem cells into and around the lungs as a treatment for Chronic Obstructive Pulmonary Disease.

22. The treatment system of claim 11, further comprising injecting the rejuvenated adult stem cells into the general circulation as a treatment for frailty and muscle dysfunction.

23. The treatment system of claim 11, further comprising injecting the rejuvenated adult stem cells into damaged or decellularized organs to regenerate the organ for transplantation into mammals.
